(19) United States
(12) Patent Application Publication
(10) Pub. No.: US 2006/0269525 A1
(43) Pub. Date: Nov. 30, 2006

(54) Co-grafts for treating Parkinson's disease

(76) Inventors: Sean Lilienfeld, Sharon, MA (US); Thomas M. DiMauro, Southboro, MA (US); Mohamed Attawia, Canton, MA (US); Chantal Holy, Raynham, MA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/137,300

(22) Filed: May 25, 2005

(51) Int. Cl. A61K 38/18; A61K 35/30

(52) U.S. Cl. 424/93.7; 514/12

(57) ABSTRACT

This invention relates to a graft for treating Parkinson's Disease, wherein, in one embodiment, the graft contains an effective amount of both a) adrenal chromaffin cells (ACCs), and b) olfactory ensheathing cells (OECs), and GDF-5.

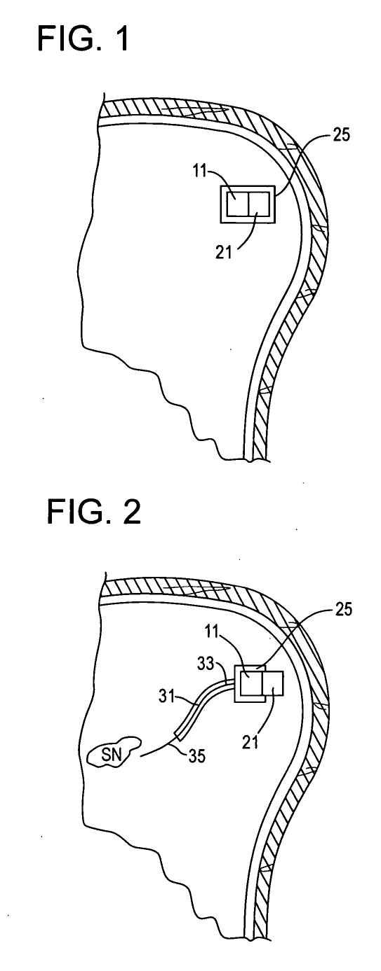

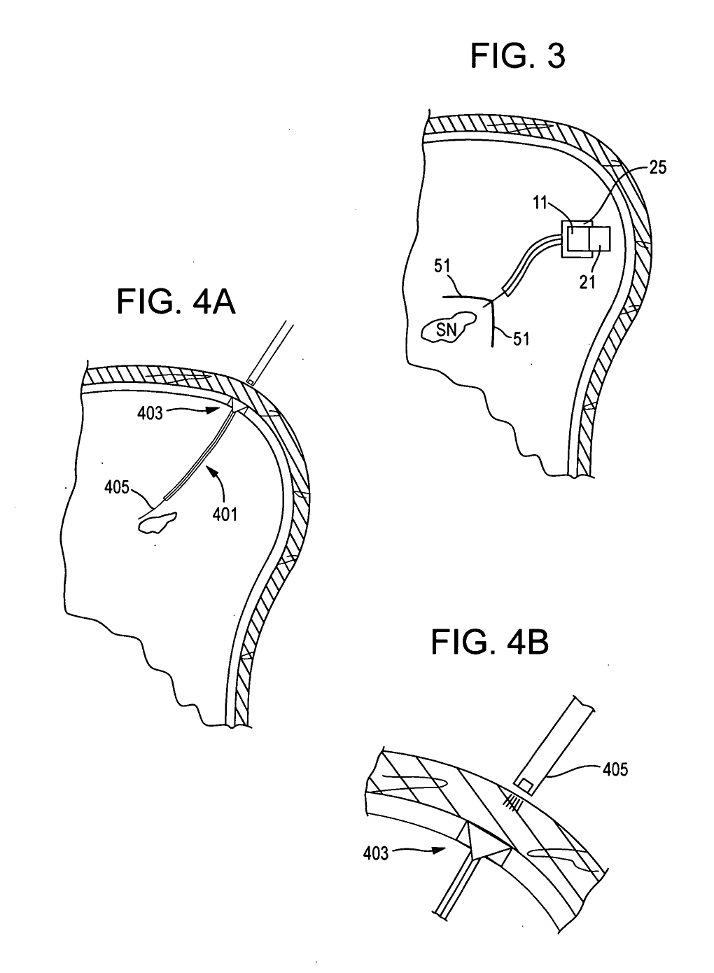

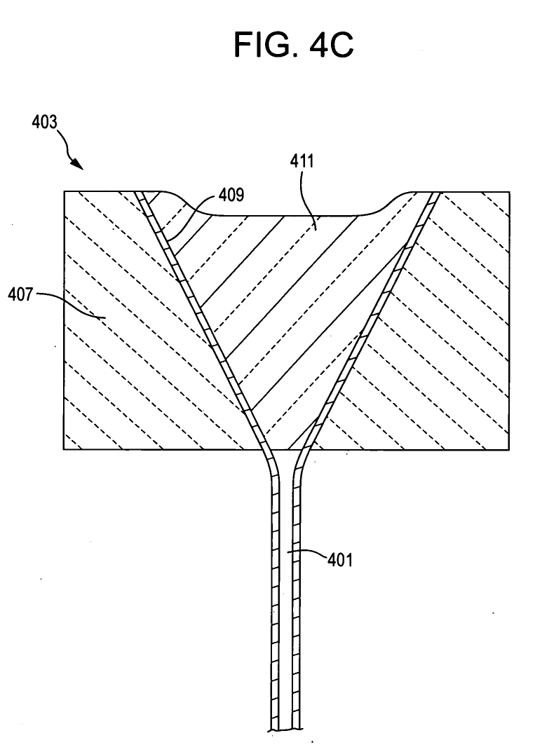

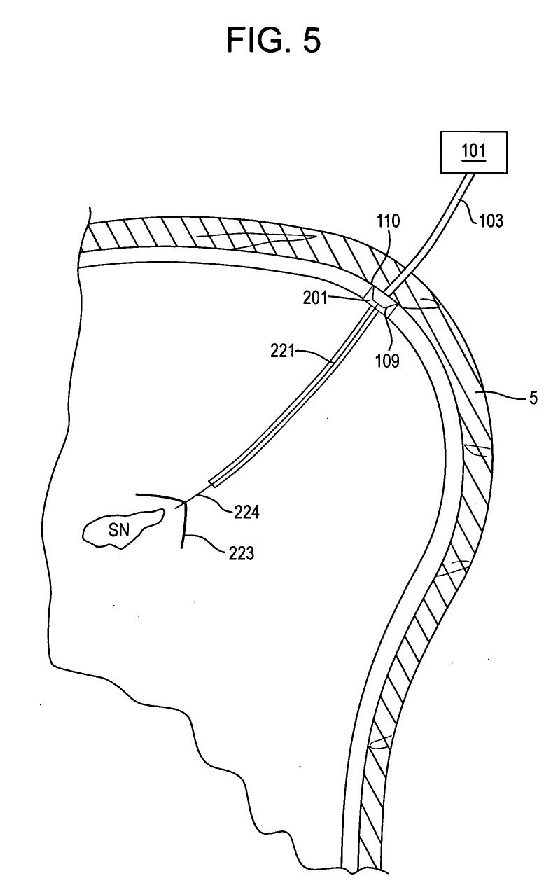

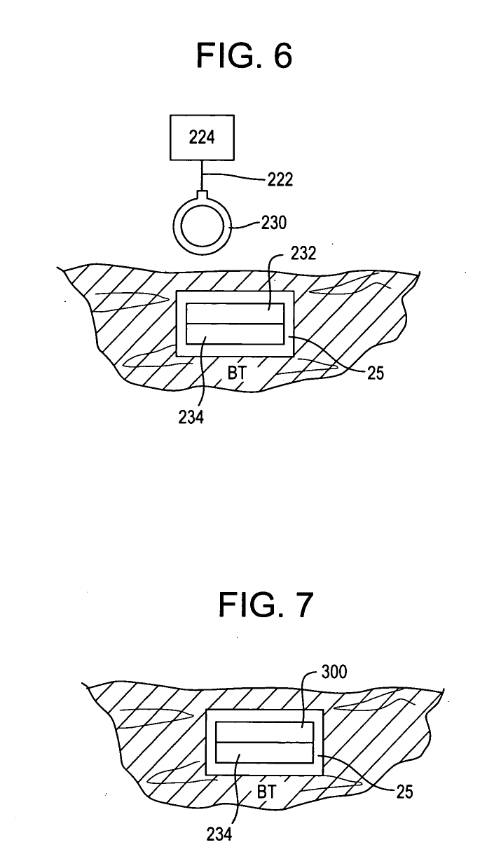

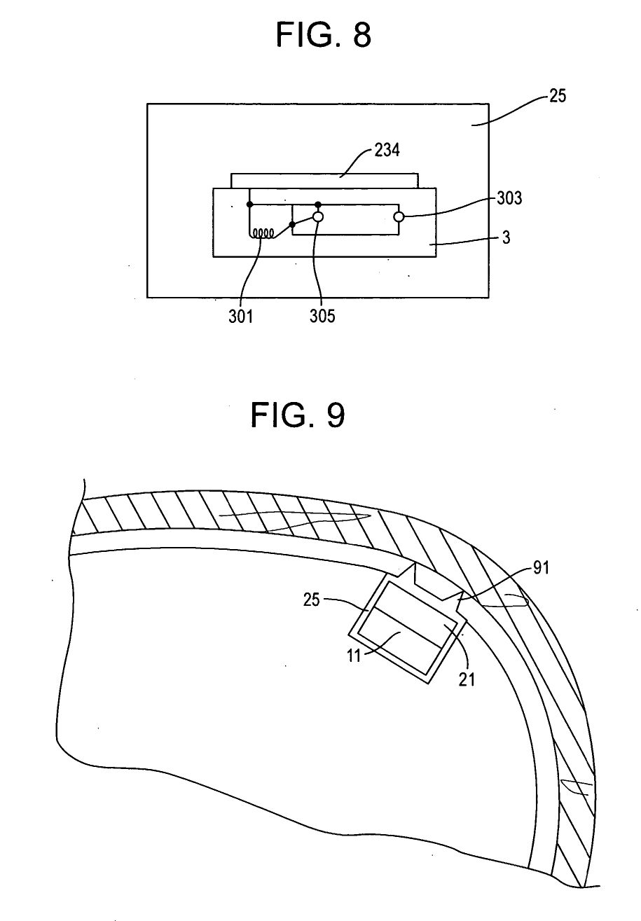

### BACKGROUND OF THE INVENTION

Parkinson's Disease is characterized by a loss of dopaminergic neurons. Attempts to replace these neurons with a graft containing dopamine-containing Adrenal Chromaffin Cells (ACCs) or fetal mesencephalic cells have been plagued by poor cell survival rates.

There appears to be an understanding that the survival rate of ACCs is increased by nerve growth factor (NGF). Some investigators have reported higher ACC survival rates when mini-pumps that emit NGF are also implanted with the ACC grafts. However, these pumps last only a few months at best. After the pumps are removed, the ACCs receive no NGF support.

### SUMMARY OF THE INVENTION

In a first aspect of the present invention, there is provided a graft for treating Parkinson's Disease, wherein the graft comprises:

a) dopaminergic cells selected from the group consisting of adrenal chromaffin cells, sympathetic ganglia cells, and stem cells having the ability to differentiate into dopaminergic cells, and

b) neurotrophic cells selected from the group consisting of olfactory ensheathing cells and glomus cells from the carotid body.

In another aspect of the present invention, there is provided a graft for treating Parkinson's Disease, wherein the graft comprises:

a) dopaminergic cells selected from the group consisting of adrenal chromaffin cells, sympathetic ganglia cells, and stem cells having the ability to differentiate into dopaminergic cells, and

b) neurotrophic cells selected from the group consisting of olfactory ensheathing cells and glomus cells from the carotid body, and

c) a growth factor selected from the group consisting of GDF-5 and at least one growth factor obtained from platelet releasate.

### DETAILED DESCRIPTION OF THE INVENTION

In a first preferred embodiment of the first aspect of the present invention, there is provided a graft for treating Parkinson's Disease, wherein the graft contains an effective amount of both a) adrenal chromaffin cells (ACCs), and b) olfactory ensheathing cells (OECs). The OECs produce and emit a continuous supply of NGF, thereby providing sustained support to the dopaminergic ACCs.

It is well known that ACC survival is increased by an exogenous supply of NGF. Hansen, Microscopy Res. Tech., 29, (1994) 155-160. OECs express large amounts of NGF, BDNF and GCDF (Woodhall, Mol. Brain. Res. 88, 203-213 (2001). Woodhall further reports that OECs release about 7 times as much NGF as BDNF. Therefore, OECs can be considered to be a NGF pump for supporting the ACCs. OECs have been successfully used in Parkinson's co-grafts precisely because of their release of the above-cited supporting factors Agrawal, Neurobiol. Disease. 16(2004) 516-24.

In addition, there are many other characteristics of OECs that could improve axonal regeneration. Three main explanations are currently being proposed. First, as noted above, they produce and release several neural growth factors, including FGF1 (Key et al., 1996), FGF2 (Chuah and Teague, 1999), NGF, BDNF, GDNF (Woodhall et al., 2000), and GGF2 (Chuah et al., 2000). OECs also express NGFr, GFRa-1 and GFRa-2, which are the receptors for NGF, GDNF, and NTN, respectively (Woodhall et al., 2001). Secondly, OECs express laminin, an extracellular matrix molecule (Treloar et al., 1996), which promotes extension of neurites, and may be involved in the migration of OEC (Tisay and Key, 1999). Third, they release two cell adhesion molecules: N-CAM (Miragall et al., 1988) and L1 (Kafitz and Greer, 1997), which support axonal elongation. OECs further assist axonal growth by secreting Semaphorin-3A, a chemorepulsion molecule that guides growing axons to their targets (Pasterkamp et al., 1998).

OEC proliferation is stimulated by bFGF in a dose-dependent manner. (Newmann, Neuroscience, 99(2), 343-50, 2000) and Chuah, Neuroscience, 88(4), 1043-50, 1999). ACCs express large amounts of bFGF and have been successfully used in Parkinson's co-grafts precisely because of their release of bFGF. Schumm, Exper. Neurology, 185 (2004) 133-42. Therefore, ACCs can be considered to be a bFGF pump for the OECs.

Therefore, co-grafted AECs and OECs will function in a mutually beneficial, autocrine manner.

It further appears that large amounts of FGF are stored in the ACC extracellular matrix. In some embodiments, this FGF is retrieved from the ACC ECM when the ACCs are isolated from the adrenal medulla.

A significant concern in human OEC transplantation is finding an accessible source of cells. The literature has showed that the olfactory bulbs and epithelium of rodents are proportionally much bigger than those of people. Therefore, biopsied human OECs are typically grown in vitro to obtain an adequately large population of cells for transplantation. Furthermore, most if not all of the past studies of human OEC transplantation have obtained cells from the olfactory bulbs. One of the great clinical appeals of OEC transplantation is the easily accessible source of OECs in the olfactory epithelium. A bulbectomy may severely disrupt the patient's sense of smell, but samples of the olfactory epithelium may be removed without behavioral consequences (see Franklin, 2002). Moreover, in that, in many instances, patients having PD already have lost their sense of smell, the procedure is even less disruptive.

However, the literature has recognized that OECs obtained from the olfactory epithelium are not plentiful and so has consistently cultured OECs for at least about 5 days in EGF or neurotrophic factors prior to their use in grafts. Therefore, in some embodiments, the OECs obtained from the epithelium are cultured ex vivo for a period of at least about 5 days.

In order to avoid this 5-day delay, in some embodiments, there is provided a short term, in vivo “culture” of the OECs. In particular, there is provided a “culture” of the OECs in vivo by providing the OECs in a sustained release device (such as fibrin gel) and exposing the OECs to a physiologically significant amount of a neurotrophic factor, EGF or IGF-II provided in the graft. The literature states that culturing OECs in EGF-enriched media for 5 days provides enough proliferation to enable the use of the OECs. The fibrin gel would keep the EGF and OECs in close proximity for five days while preventing ingress of fibroblasts. After a few days, the OECs will have proliferated to a large amount and the fibrin gel would have eroded away. In some embodiments, autologous cryoprecipitated fibrinogen is used to provide a longer lasting in vivo culture.

In other embodiments, a biodegradable encapsulation device (such as a protective shell made of PLA, PGA or PCL) is used to exclude native fibroblasts from the growth factors. After a few days, the shell would erode away. In other embodiments, we could use a synthetic, temporary, semi-permeable membrane that lets in water and nutrients and keeps out fibroblasts.

In a second preferred embodiment of the first aspect of the present invention, glomus cells from the carotid body are selected as the neurotrophic cells. Glomus cells appear to release large amounts of GDNF, a neurotrophic factor believed to provide support to dopamine neurons in the host. Preferably, the glomus cells are combined with adrenal chromaffin cells.

Therefore, there is provided a graft for treating Parkinson's Disease, wherein the graft contains an effective amount of both a) adrenal chromaffin cells, and b) glomus cells. The glomus cells emit a continuous supply of GDNF, thereby providing sustained support not only to the dopaminergic ACCs, but also to the host dopamine neurons.

In other embodiments, cells genetically engineered to release NGF, bFGF, of both may be used as a replacement of OECs or ACCs.

In some embodiments, the dopaminergic cells are stem cells. The stem cells of the present invention can be any stem selected capable of differentiating into a dopaminergic cell. In some embodiments, the stem cells are fetal stem cells. In other embodiments, the stem cells are autologous and are taken from either the bone marrow or adipose tissue of the patient.

In some embodiments, the co-graft is a suspension or pellet having a volume of between about 0.1 cc and about 2 cc, and is preferably between 0.5 and 1.5 cc. Typically, the graft has a dopaminergic cell density of 100,000 cells/cc and 2 million cells/cc (preferably between 200,000 cells/cc and 1 million cells/cc). Typically, the graft has a neurotrophic cell density of 100,000 cells/cc and 2 million cells/cc (preferably between 200,000 cells/cc and 1 million cells/cc).

In some embodiments, either a) GDF-5, or b) growth factors obtained from autologous platelet releasate, or c) autologous VEGF are combined with the recited dopaminergic cells or neurotrophic cells. Therefore, in a second aspect of the present invention, there is provided a graft for treating Parkinson's Disease, wherein the graft comprises:

a) neurotrophic cells selected from the group consisting of olfactory ensheathing cells and glomus cells from the carotid body, and

b) a growth factor selected from the group consisting of GDF-5 and at least one growth factor obtained from platelet releasate.

In a third aspect of the present invention, there is provided a graft for treating Parkinson's Disease, wherein the graft comprises:

a) dopaminergic cells selected from the group consisting of adrenal chromaffin cells, sympathetic ganglia cells, and stem cells having the ability to differentiate into dopaminergic cells, and

b) a growth factor selected from the group consisting of GDF-5 and at least one growth factor obtained from platelet releasate.

GDF-5 has been shown to have neurotrophic and neuroprotective effects on dopaminergic neurons both in vitro and in vivo. Sullivan, Eur J Neurosci. 1998 Dec;10(12):3681-8.) reports investigating the effects of GDF-5 on foetal mesencephalic grafts transplanted into a rat model of Parkinson's disease, and comparing them with those of glial cell line-derived neurotrophic factor. This study showed that GDF-5 is at least as effective as glial cell line-derived neurotrophic factor (GDNF) in enhancing the survival and functional activity of mesencephalic grafts, and thus is an important candidate for use in the treatment of Parkinson's disease.

In some embodiments, GDF-5 is provided in the graft at a concentration of about 1 ng/ml to about 100 ug/ml, preferably between about 5 ng/ml and 50 ng/ml.

In some embodiments, GDF-5 and TGF-μ are combined. These factors have a synergistic neurotrophic activity. See USP 6,790,824, the specification of which is incorporated by reference in its entirety. The TGF-μ can be derived from platelet releasate. TGF-B can be isolated from platelet releasate by binding to a heparin affinity column and then elution with 0.9-1.2 M NaCl.

Many investigators have reported that IGF-I enhances the proliferation of cells used in Parkinson's grafts. Yan, Glia, Mar 15, 33(4), 334-42 (OECs) and Frodin, PNAS, Mar. 1, 1994, 91(5), 1771-5. Frodin reported using a 10 nM IGF-I concentration in the culture. Clarkson, Exp. Neurol., 2001 March, 168(1) 183-91 reports that 150 ng/ml IGF-I was neurotrophic. Therefore, in some embodiments, the graft contains between about 10 and 1000 ng/ml IGF-I, preferably obtained from platelet releasate.

It is further known that EGF behaves as a mitogenic regulator for stem cells populations from the SVZ portion of the forebrain that contribute new neurons to the olfactory bulb. Gritti, J. Neuroscience, May 1, 1999, 19(9) 3287-97. Newman, Neuroscience 99(2) 343-350, 2000 reported using 25 ng/ml EGF as the primary proliferative growth factor in a culture for OECs. Therefore, in some embodiments, the graft contains between about 1 and 100 ng/ml EGF, preferably obtained from platelet releasate.

Newman, Neuroscience 99(2) 343-350, 2000 reported that 0.1-100 ng/ml PDGF provided for enhanced survival of OECs in culture, with the best effect seen at about 1 ng/ml. Therefore, in some embodiments, the graft contains between about 0.1 and 10 ng/ml PDGF, preferably obtained from platelet releasate.

Schanzer, Brain Pathol., 2004, 14, 237-248 reports that VEGF provided both a beneficial proliferative and survival effect upon neural stem cells derived from the lateral wall of the lateral ventrical system of rats. Expansion increased 2-2.5X over control when VEGF was provided in the range of 10-100 ng/ml. Apoptosis decreased from 100% to about 70% -80% when 10-100 ng/ml VEGF was added. Therefore, in some embodiments, the graft contains between about 10 and 100 ng/ml VEGF, preferably obtained from platelet releasate.

Newman, Neuroscience 99(2) 343-350, 2000 reported that 1-20 ng/ml TGF-β provided for differentiation of OECs. Therefore, in some embodiments, the graft contains between about 1 and 20 ng/ml TGF-β, preferably obtained from platelet releasate.

The approximate concentrations found in about 10 cc's of platelet releasate, based upon about 10⁶ platelets, are as follows:

| | |
|---|---|
| IGF-I | 100 ng/ml |
| PDGF | 100 ng/ml |
| EGF | 1 ng/ml |
| VEGF | 1 ng/ml |
| TGF-β | 100 ng/ml |

The low concentrations reported here are provided in the context of a spinal bone graft, and are believed to be due to the dilution with PPP. Because a graft for Parkinson's Disease is typically only about 0.1 cc, the concentrations realizable in a Parkinson's graft derived from PRP are about 100X higher, or as follows:

| | |
|---|---|
| IGF-I | 10000 ng/ml |
| PDGF | 10000 ng/ml |
| EGF | 100 ng/ml |
| VEGF | 100 ng/ml |
| TGF-β | 10000 ng/ml |

When the levels obtained in a 0.1 cc volume of platelet releasate are compared to those found to be physiologically beneficial to Parkinson's graft cells, it is clear that platelet releasate may be a very valuable source of growth factors for Parkinson's grafts.

In some embodiments, it may be beneficial to add only selected growth factors from the platelet releasate to the graft. For example, since TGF-β is known to prevent mitosis of ACCs, it may be useful to either exclude TGF-β from a ACC-containing graft, or to provide it in a sustained release device so that it can be released after ACC mitosis has occurred.

It has been observed that most of the growth factors present in platelet releasate can be bound by heparin, and then eluted with NaCl.

| Growth | NaCl | |
|---|---|---|
| Factor | Strength | Source |
| IGF-I | No Binding | Ikezawa, Conn. Tiss. Res., |
| | | 1997, 36(4) 309-319 |
| PDGF | 0.5 | Mangrulkar, Biol. Repro. |
| | | 1995, Sept. 52(3), 636-46 |
| EGF | 1.1* | Mangrulkar, Biol. Repro. |
| | | 1995, Sept. 52(3), 636-46 |
| VEGF | 0.5 | Sakurada, Jpn. J. Can. Res, |
| | | 1996, Nov. 87, (11), 1143-52 |
| TGF-β | 0.9-1.2 | McCaffrey, J. Cell. Phys. |
| | | 1992, Aug., 152(2) 430-40 |

*EGF (6 kDa) is a much smaller molecule than the other listed growth factors (˜25-35 kDa), and so is often separated from other growth factors via size exclusion filters.

Therefore, we can selectively isolate different growth factors or sets or growth factors from platelet releasate and then combine them in different phases for predetermined delivery to the cells.

Because it might be beneficial to separate the release of the proliferative factor from the differentiating factor (e.g., TGF-B stops ACC proliferation), in some embodiments, the proliferative factors are provided in a first phase (e.g., conventional fibrin gel), while other factors (such as TGF-B) are provided in a sustained release fibrin gel (such as a cryoprecipitated fibrin gel). The cryoprecipitated fibrin will have a much higher concentration and so will not degrade as quickly as conventional fibrin gel.

In some preferred embodiments using a growth factor from platelet releasate, adrenal cell may be used as the dopaminergic cell. In some embodiments, olfactory. ensheathing cells, which emit NGF, may be added to the graft. In some embodiments, OECs and ACCs are combined in the graft. In some embodiments, glomus cells from the carotid body may also be added to the graft. Glomus cells appear to release large amounts of GDNF, a neurotrophic factor believed to provide support to dopamine neurons in the host.

In a fourth aspect of the present invention , there is provided a graft for treating Parkinson's Disease, wherein the graft comprises:

a) dopaminergic cells, and

b) autologous VEGF.

In some embodiments, autologous VEGF is provided by contacting macrophages obtained from the patient's blood with reactive oxygen species (ROS). The ROS induce VEGF production by the macrophages. The induced macrophages are then added to the graft.

In some embodiments thereof, the inducer is a low concentration of reactive oxygen species (ROS). Cho, Am. J. Physiol. Heart Circ. Physiol. 1280, 2001, H2357-63, reports that VEGF RNA and VEGF protein release are induced by hydrogen peroxide in human macrophages. In particular, Cho reported that exposing macrophages to a 0.5 mM concentration of H₂O₂ for about 30 minutes produces about 2.5 fg VEGF/cell. Filtration and dewatering of blood, when carried out in accordance with U.S. Pat. No. 5,733,545 (Hood), provides a buffy coat having about 14×106 monocytes/ml. Inducing 1 ml of this monocyte-rich buffy coat with 1 ml H₂O₂ should produce about 2 ml of 17 ng/ml VEGF (based upon Cho's production rate of 2.5 fg VEGF/ cell). The 17 ng/ml VEGF concentration in this mixture is about 50 times that of whole blood. In other embodiments thereof, the reactive oxygen species (ROS) are produced via photocatalytic oxidation.

Brauchle, J. Biol. Chem., 271, 36, 21793-97, 1996, reports a strong superinduction effect provided by cycloheximide to human keratinocytes cultured in H₂O₂. The level of MRNA produced by the superinduction appear to be at least about 10 fold higher than that produced by the addition of H₂O₂. Therefore, in some embodiments, the macrophages are contacted with a device having a cyclohexamine coating.

In a fifth aspect of the present invention, there is provided a graft for treating Parkinson's Disease, wherein the graft comprises:

a) cells selected from the group consisting of dopaminergic cells and neurotrophic cells, and

b) an anti-oxidant selected from the group consisting of catalase, superoxide dismutase (SOD) and Vitamin E.

In some embodiments, Catalase, SOD and/or Vitamin E are combined in the graft as anti-oxidant support.

Oxidative stress is a major component in the onset of Parkinson's Disease. It is believed that the substantia nigra contains high levels of iron, which helps catalyze oxygen to reactive oxygen species (ROS). In addition, it is believed that the dissection and manipulation of cells during graft preparation leads to the production of ROS.For these reasons, it is believed that ROS significantly contribute to the low survival rate of transplanted cells.

The literature describes the general use of anti-oxidants as potent neuroprotective agents for Parkinson's disease. For example, Dugan, Parkin. Relat. Disord., 1002, Jul 7, (3) 243-6, describes the use of fullerene-based antioxidants as neuroprotective drugs. In addition, oral administration of ascorbic acid has been tried as a therapy for Parkinson's Disease. The literature describes adding selected anti-oxidants to grafts for the treatment of PD. For example, Hansson, Exp. Neurol. 164, 102-111 (2000) describes the use of lazaroids (an anti-oxidant) and a caspase inhibitor with a nigral DA graft. Nakao, PNAS, 91, Dec. 1994, 12408-12 describes the use of lazaroids with an embryonic dopamine graft. Love, Cell Transplant., 2002, 11(7), 653-62 describes the use of NAC, glutathione (GSH) and lazaroids with a graft of DA neurons. Agrawal, J. Chem. Neuroanatomy, 28 (2004) 253-264 describes the use of GSH and ascorbic acid with a graft of dopaminergic neurons (ventral mesencephalic cells and nigral dopamine neurons).

None of these publications teach the use of either catalase, SOD or Vitamin E as an anti-oxidant with grafts as a treatment for Parkinson's Disease.

Therefore, in some embodiments of the present invention, there is provided a graft containing either dopaminergic cells or neurotrophic cells combined with an anti-oxidant selected from the group consisting of catalase, SOD or Vitamin E.

Gonzalez, Cell Biology Int'l, 28(2004) 373-80, clearly reports the effectiveness of catalase in enhancing the viability of PD cells in culture. In particular, Gonzalez reports that 50 U/ml catalase increased the in vitro viability of cerebellar granule cells exposed to MPP+ neurotoxin from about 50% to about 75%.

Furthermore, it is believed that catalase is superior to the anti-oxidants recited in the prior art because it is not only an anti-oxidant, it is also neurotrophic towards CNS neurons. See Wallicke, J. Neuroscience, April 1986, 6(4), 1114-21.

Therefore, in some embodiments, the graft contains between about 5 and 500 U/ml of catalase.

SOD has been shown to be neuroprotective in a rat model of Parkinson's disease. Nakao Nat. Med. 1995, Mar 1, (3) 226-231, reports that the survival of grafted dopaminergic neurons in transgenic rats designed to overexpress Cu/Zn SOD was about four times higher than those in control rats, and there was also a similar increase in functional recovery.

It is believed that Vitamin E would also be a superior anti-oxidant support for grafted cells. Gonzalez, Cell Biology Int'l, 28(2004) 373-80, clearly reports the effectiveness of Vitamin E in treating PD cells in culture. In particular, Gonzalez reports that 100 μM Vitamin E increased the in vitro viability of cerebellar granule cells exposed to MPP+ neurotoxin from about 50% to about 85%. Vitamin E appeared to be the most protective anti-oxidant tested by Gonzalez. Therefore, in some embodiments, the graft has a Vitamin E concentration of between about 10 and 1000 μM.

The catalase, SOD or Vitamin E may be exogenous of autologous. Human erythrocyte catalase (Cat. No. A50136H) is available from Biodesign International, Saco, Me.

In some embodiments, the catalase can be obtained from an autologous hemolysate, as decribed in Wallicke, J. Neuroscience, April 1986, 6(4), 1114-21. Hemolysate is known to have neurotrophic qualities. Wallicke basically separates this enzyme from hemoglobin and other proteins in the hemolysate by DEAE affinity chromatography. The column is then repeated washed with water, and then the catalase still bound to the DEAE column is then eluted with 0.035 M NaCl. This process looks like a simple and safe process that can be performed in the operating theatre.

In another interesting embodiment, each of GSH, catalase and SOD can be obtained from autologous hemolysate as taught by Stepanik, J. Biochem. Biophys. Methods, 20, (1990) 157-69. Stepanik basically co-isolates these three enzymes from hemoglobin and other proteins by DEAE affinity chromotography, and then elutes them with NaPO₄. We can practice the isolation steps of Stepanik and then add the isolate to the selected graft to treat PD.

In some embodiments, at least one of GSH, catalase and SOD is obtained by applying electrophoresis to hemolysate having its hemoglobin removed.

In some embodiments, autologus hemolysate (having its hemoglobin removed) is used as the anti-oxidant. Wallicke, supra, reports that hemolysate was effective in providing neurotrophic support to chick forebrain neurons in vitro. Williams, Brain. Research., 336 (1985) 99-105 also reports the neurotrophic activity of red blood cell extract on E8 chick forebrain neurons.

In some embodiments using anti-oxidants, these anti-oxidants are used to support grafts made of adrenal chromaffin cells and olfactory ensheathing cells. In some embodiments, these anti-oxidants are used to support grafts made of glomus cells of the carotid body. In some embodiments, these anti-oxidants are used to supports grafts made of adrenal chromaffin cells, olfactory ensheathing cells, and glomus cells.

According to Olanow, Trends. Neurosci., (1996), 19, 102-109, functional benefits following grafting are site-specific. Grafts placed in the dorsal striatum of the patient ameliorate rotational asymmetries. Therefore, in some embodiments, the graft is placed in the dorsal striatum. Grafts placed within the lateral striatum improve sensori-motor symptoms. Therefore, in some embodiments, the graft is placed in the lateral striatum. Grafts placed into the caudate nucleus improve rotation. Therefore, in some embodiments, the graft is placed in the caudate nucleus. Grafts placed into the putamen improve spontaneous contralateral limb use. Therefore, in some embodiments, the graft is placed in the putamen. Olanow indicates that the posterior putamen may be a primary target of the graft, as microstimulation studies within the posterior putamen elecit discrete movements of contralateral body parts, and autopsy studies has demonstrated greater dopamine depletion in the posterior putamen than the in the anterior putamen/caudate nucleus complex. Therefore, in some embodiments, the graft is placed in the posterior putamen.

According to Olanow, Trends. Neurosci., (1996), 19, 102-109, limited dopamine diffusion implies that it might be desirable to distribute cells at 5 mm intervals through the three-dimensional configuration of the target region. This can be accomplished in the posterior putamen or in the anterior putamen caudate with six to eight needle tracts containing four deposits per tract.

In some embodiments, the graft is deposited into both the posterior (post-commissural) putamen and the anterior putamen/caudate nucleus complex.

In some embodiments, the patient can receive a unilateral transplant, while in other the patient can receive bilateral transplant.

### EXAMPLE

This prophetic example describes how to implant a preferred co-graft of the present invention into a patient suffering from Parkinson's Disease:

1. Obtaining Autologous ACCs

ACCs may be obtained from the patient in a manner similar to that described in Schumm, Exp. Neurology, 185(2004)133-142. Briefly, ACCs are obtained from the patient and isolated using routine procedures. A portion of the medullary tissue is removed from the cortical tissue. The removed portion is then minced, filtered and plated in DMEM/F12 with 10% fetal bovine serum (Hyclone). The cells are then differentially plated to remove fibroblasts and to further purify the preparation. Isolated ACCs are then resuspended in Hank's Buffered Salt Solution (HBSS, Sigma) before grafting.

2. Obtaining Autologous OECs

OECs may be obtained from the patient in a manner similar to that described in Agrawal, Neurobiol. Disease, 16 (2004) 516-26. Briefly, OECs are obtained from the glomerular region of the patient's olfactory bulb or from the epithelium of olfactory nerves extending from the bulb. The olfactory nerve fiber tissue is removed, washed twice with HBSS, diced into small fragments, and incubated with 0.1% trypsin at 37° C. for 15 minutes. Trypsinization is stopped by adding DMEM:F12 medium supplemented with 10% fetal bovine serum. The OEC cell suspension is then centrifuged at 800 rpm for 3 min, resuspended in DMEM:F12 medium with 10% serum, and this step is repeated twice. Cell dissociation is achieved by passing the tissue 15-20 times through a fire polished siliconized Pasteur pipette. Viable cells, in the form of a single cell suspension, are then plated in culture flasks precoated with PLL at a density of 6.5×106 per flask and maintained in DMEM:F12 supplemented with 2 mM glutamine, 10,000 U/ml penicillin, 10,000 μg/ml streptomycin, and 25 ug/ml amphoterecin B. The OEC culture is then maintained in 5% CO₂ atmosphere at 37° C. and the medium is changed every two days. After 14 days in culture, the cells are trypinized and centrifuged for 10 minutes at 100 g after being neutralized with DMEM:F12 supplemented with 10% FBS. The OEC cells are then resuspended in DMEM:F12 at a density of 125,000 cells/cc.

3. Co-Culture

Cells for co-grafts are prepared by combining equal volumes of OECs and ACCs and gently titrating the suspension. The OECs and ACCs obtained above are allowed to grow in a co-culture environment for about 5 days, or until the appropriate cell densities are reached. Alternatively, the cells are cultured separately and then mixed together.

4. Mix with GDF-5

Mixing GDF-5 with the cell suspensions may be carried out in accordance with the procedure described in Sullivan, Eur. J. Neuroscience, 10, 3681-8 (1998). Briefly, the co-cultured cell suspension obtained above is placed in a tube and recombinant human GDF-5 (in a buffer comprising 10 mM citrate and 150 mM sodium chloride) is added to the tube. The final GDF-5 concentration of the graft is 20 ng/ml.

5. Grafting

Grafting may be performed in a manner similar to that described in Schumm, ExD. Neurology, 185(2004)133-142. Before grafting, the graft cells are spun down and resuspended in a microcentrifuge tube in HBSS and kept on ice. The patient is anesthetized and placed in a stereotaxic apparatus. Surgical coordinates may be determined relative to the bregma and dura. A saggital incision is made in the scalp and a burr hole in drilled into the skull without breaking the dura. A 25 gauge Hamilton syringe is lowered into the striatum, and allowed to remain in position or several minutes. 800 microliters of suspension are injected and the syringe is slowly raised as the cells are slowly injected. ACCs are implanted at a density of 100,000 cells/cc, and OECs are implanted at a density of 125,000 cells /cc. The co-grafts contained 1 cc of each cell type. Thus, the co-graft contained about 100,000_ACCs and 125,000 OECs. The incision is then closed with a nylon suture.

Because a bilateral transplant is preferred, the grafting procedure described above is repeated at a second site in the striatum.

We claim:

1. A graft for treating Parkinson's Disease, wherein the graft comprises:
a) dopaminergic cells selected from the group consisting of adrenal chromaffin cells, sympathetic ganglia cells, and stem cells having the ability to differentiate into dopaminergic cells, and
b) neurotrophic cells selected from the group consisting of olfactory ensheathing cells and glomus cells from the carotid body.

2. The graft of claim 1 wherein the dopaminergic cells are adrenal chromaffin cells.

3. The graft of claim 1 wherein the dopaminergic cells are sympathetic ganglia cells.

4. The graft of claim 1 wherein the dopaminergic cells are stem cells having the ability to differentiate into dopaminergic cells.

5. The graft of claim 1 wherein the neurotrophic cells are olfactory ensheathing cells.

6. The graft of claim 1 wherein the neurotrophic cells are glomus cells from the carotid body.

7. The graft of claim 1 wherein the dopaminergic cells are adrenal chromaffin cells, and the neurotrophic cells are olfactory ensheathing cells.

8. The graft of claim 1 wherein the dopaminergic cells are adrenal chromaffin cells, and. the neurotrophic cells are glomus cells from the carotid body.

9. The graft of claim 1 wherein the dopaminergic cells are stem cells having the ability to differentiate into dopaminergic cells, and the neurotrophic cells are olfactory ensheathing cells.

10. The graft of claim 1 wherein the neurotrophic and dopaminergic cells are co-cultured.

11. A graft for treating Parkinson's Disease, wherein the graft comprises:
a) dopaminergic cells selected from the group consisting of adrenal chromaffin cells, sympathetic ganglia cells, and stem cells having the ability to differentiate into dopaminergic cells, and
b) a growth factor selected from the group consisting of GDF-5 and at least one growth factor obtained from platelet releasate.

12. The graft of claim 11 wherein the dopaminergic cells are adrenal chromaffin cells.

13. The graft of claim 11 wherein the dopaminergic cells are sympathetic ganglia cells.

14. The graft of claim 11 wherein the dopaminergic cells are stem cells having the ability to differentiate into dopaminergic cells.

15. The graft of claim 11 wherein the growth factor is GDF-5.

16. The graft of claim 11 wherein the growth factor is obtained from platelet releasate.

17. The graft of claim 11 wherein the dopaminergic cells are adrenal chromaff-m cells, and the growth factor is GDF-5.

18. The graft of claim 11 wherein the dopaminergic cells are sympathetic ganglia cells, and the growth factor is GDF-5.

19. The graft of claim 11 wherein the dopaminergic cells are stem cells having the ability to differentiate into dopaminergic cells, and the growth factor is GDF-5.

20. The graft of claim 11 further comprising cells genetically engineered to release NGF.

21. A graft for treating Parkinson's Disease, wherein the graft comprises:
a) neurotrophic cells selected from the group consisting of olfactory ensheathing cells and glomus cells from the carotid body, and
b) a growth factor selected from the group consisting of GDF-5 and at least one growth factor obtained from platelet releasate.

22. The graft of claim 1 wherein the neurotrophic cells are olfactory ensheathing cells.

23. The graft of claim 21 wherein the neurotrophic cells are glomus cells from the carotid body.

24. The graft of claim 21 wherein the growth factor is GDF-5.

25. The graft of claim 21 wherein the growth factor is at least one growth factor obtained from platelet releasate.

26. The graft of claim 1 wherein the neurotrophic cells are olfactory ensheathing cells and the growth factor is GDF-5.

27. The graft of claim 21 wherein the neurotrophic cells are glomus cells from the carotid body and the growth factor is GDF-5.

28. The graft of claim 21 further comprising cells genetically engineered to release NGF.

29. The graft of claim 11 further comprising cells genetically engineered to release bFGF.

30. A graft for treating Parkinson's Disease, wherein the graft comprises:
a) cells selected from the group consisting of dopaminergic cells and neurotrophic cells, and
b) an anti-oxidant selected from the group consisting of catalase, superoxide dismutase (SOD) and Vitamin E.

31. The graft of claim 31 wherein the cells are dopaminergic cells.

32. The graft of claim 32 wherein the anti-oxidant is catalase.

33. The graft of claim 32 wherein the anti-oxidant is SOD.

34. The graft of claim 32 wherein the anti-oxidant is Vitamin E.

35. The graft of claim 31 wherein the cells are neurotrophic cells.

36. The graft of claim 35 wherein the anti-oxidant is catalase.

37. The graft of claim 35 wherein the anti-oxidant is SOD.

38. The graft of claim 35 wherein the anti-oxidant is Vitamin E.

39. The graft of claim 1 wherein the anti-oxidant is autologous.

40. A graft for treating Parkinson's Disease, wherein the graft comprises:
a) dopaminergic cells selected from the group consisting of adrenal chromaffin cells, sympathetic ganglia cells, and stem cells having the ability to differentiate into dopaminergic cells, and
b) neurotrophic cells selected from the group consisting of olfactory ensheathing cells and glomus cells from the carotid body, and
c) a growth factor selected from the group consisting of GDF-5 and at least one growth factor obtained from platelet releasate.

41. The graft of claim 40 wherein the dopaminergic cells are adrenal chromaffin cells.

42. The graft of claim 40 wherein the dopaminergic cells are sympathetic ganglia cells.

43. The graft of claim 40 wherein the dopaminergic cells are stem cells having the ability to differentiate into dopaminergic cells.

44. The graft of claim 40 wherein the neurotrophic cells are olfactory ensheathing cells.

45. The graft of claim 40 wherein the neurotrophic cells are glomus cells from the carotid body.

46. The graft of claim 40 wherein the growth factor is GDF-5.

47. The graft of claim 40 wherein the growth factor is obtained from platelet releasate.

48. The graft of claim 40 wherein the dopaminergic cells are adrenal chromaffin cells, and the neurotrophic cells are olfactory ensheathing cells.

49. The graft of claim 40 wherein the dopaminergic cells are adrenal chromaffin cells, and the growth factor is GDF-5.

50. The graft of claim 40 wherein the neurotrophic cells are adrenal chromaffin cells and the growth factor is GDF-5.

51. The graft of claim 40 wherein the dopaminergic cells are adrenal chromaffin cells, and the neurotrophic cells are olfactory ensheathing cells, and the growth factor is GDF-5.
